(19) United States
(12) Patent Application Publication
(10) Pub. No.: US 2009/0170947 A1
(43) Pub. Date: Jul. 2, 2009

(54) DISINFECTANT COMPOSITIONS, METHODS AND SYSTEMS

(75) Inventors: Dinesh Aggarwal, Franklin, MA (US); Chirag Shah, N. Attleboro, MA (US)

Correspondence Address: BUCHANAN INGERSOLL & ROONEY, PC, P.O. BOX 1404, ALEXANDRIA, VA, 22313-1404, US

(73) Assignee: Tyco Healthcare Group LP, Mansfield, MA (US)

(21) Appl. No.: 12/347,082

(22) Filed: Dec. 31, 2008

Related U.S. Application Data
(60) Provisional application No. 61/006,204, filed on Dec. 31, 2007.

(51) Int. Cl. A61K 31/197

(52) U.S. Cl. 514/566

(57) ABSTRACT

Disinfectant compositions comprising PHMB and EDTA salt(s) are disclosed. The disinfectant compositions have also demonstrated activity as enhanced, fast acting catheter lock/flush solutions. They are safe for human and medical uses and may be used as prophylactic preparations to prevent infection, or to reduce the proliferation of and/or eliminate existing or established infections.

### BACKGROUND

Infections are a significant problem in many fields where sanitary conditions are important, such as in healthcare. Problematic infections may arise from bacterial, fungal, amoebic, protozoan and/or viral organisms. Challenges are encountered both in preventing infection, and in reducing or eliminating the infection once it is established. Infected environments may include surfaces of objects, fluids and fluid conduits and/or humans or animals.

Alcohol solutions and isopropyl alcohol wipes are commonly used to disinfect surfaces and have been shown to have antibacterial activity. The most effective inhibitory anti-microbial effect is seen with 70% isopropanol solutions. Alcohol solutions at this concentration are quite expensive and rapidly evaporate, which substantially diminishes their efficacy and increases their cost. Moreover, although isopropanol solutions may be used for surfaces, including human skin, and in a variety of medical applications, alcohol solutions of this concentration cannot be administered to humans, for medical purposes, other than topically.

In the healthcare field, infections of various types and causes are common and often result in longer hospital stays, producing higher hospital costs. Even worse, over 90,000 patient deaths annually are attributed to nosocomial infections—that is, infections acquired at a hospital or in another healthcare environment. Surveillance for nosocomial infection has become an integral part of hospital practice. Studies conducted more than 20 years ago by the Centers for Disease Control and Prevention (CDC) documented the efficacy of these surveillance activities in reducing nosocomial infection occurrence. Despite the attention paid to problems of nosocomial infection, however, infection rates have not been dramatically reduced, and nosocomial infections remain a substantial risk and a substantial health concern.

One problematic source of infections in the medical and veterinary fields is found in catheters, and particularly in in-dwelling catheters. Catheters have become essential in the management of critical care patients, yet the inside of a catheter is often the major source of infection. Catheters are used for delivery of fluids, blood products, drugs, nutrients, hemodialysis, hemofiltration, peritoneal dialysis, retrieval of blood samples, monitoring of patient conditions, etc. Transcutaneous catheters often become infected through skin penetration of the catheter. It has been found that seventy percent (70%) of all nosocomial bloodstream infections occur in patients with central venous catheters. Daouicher et al. 340, 1-8, NEW ENGLAND JOURNAL OF MEDICINE (1999).

In particular, during some procedures, a catheter must be implanted in, and remain implanted in, a patient for a relatively long period of time, e.g. over thirty days. Intravenous (IV) therapy catheters and urinary catheters typically remain implanted for a substantial period of time. As a result of trauma to the areas of insertion, and pain to the patients, such catheters can't be removed and implanted frequently. Catheter-borne bacteria are implicated as a primary source of urinary tract infections. Patients who receive a peripherally inserted central catheter during pregnancy have also been found to be at significant risk for infectious complications. “Complications Associated With Peripherally Inserted Central Catheter Use During Pregnancy” AM. J. OBSTET. GYCOL. 188(5):1223-5 May 2003. In addition, central venous catheter infection, resulting in catheter related sepsis, has been cited as the most frequent complication during home parenteral nutrition. CLINICAL NUTRITION, 21(1):33-38, 2002. Because of the risk of infections, catheterization may be limited to incidences when the procedure is absolutely necessary. This seriously compromises patient health.

After most prescribed access medical procedures involving a catheter, the catheter is flushed with saline and then filled with a liquid, such as saline or a heparin solution, to prevent blood from clotting inside of the catheter, to inhibit the patient's blood from backing up into the catheter, and to prevent gases from entering the catheter. The liquid that is used to flush the catheter is referred to as a “lock-flush,” and the liquid used to fill the catheter following flushing or during periods of non-use is referred to as a “lock” solution.

Traditionally, catheters have been locked with normal saline or heparin solutions. Heparin and saline are sometimes used in combination. Normal saline is generally used to lock short term peripheral intravenous catheters, but saline has no anticoagulant or antimicrobial activity. Heparin solutions are generally used to lock vascular catheters. Heparin has anticoagulant activity but it does not function as an antimicrobial and does not prevent or ameliorate infections. There are also strong indications that heparin in lock solutions may contribute to heparin-induced thrombocytopenia, a serious bleeding complication that occurs in a subset of patients receiving heparin injections.

Catheter locking solutions comprising taurolidine, citric acid and sodium citrate have been proposed. A recent publication (Kidney International, September 2002) describes the use of a 70% alcohol solution as a lock solution for a subcutaneous catheter port. The use of alcohol as a lock solution is questionable, since it is not an anticoagulant, and since there would be risks associated with this solution entering the bloodstream. There is also no evidence that the inventors are aware of that indicates that a 70% alcohol solution has any biofilm eradication activity.

An emerging trend and recommendation from the Center for Infectious Disease (CID) is to treat existing catheter infections systemically with either a specific or a broad range antibiotic. Use of an antibiotic in a lock solution to prevent infection is not recommended. The use of antibiotics to treat existing catheter infections has certain risks, including: (1) the risk of antibiotic-resistant strains developing; (2) the inability of the antibiotic to kill sessile, or deep-layer biofilm bacteria, which may require the use of antibiotics at toxic concentrations; and (3) the high cost of prolonged antibiotic therapy. Catheters coated with a disinfectant or antibiotic material are available. These coated catheters may only provide limited protection for a relatively short period of time.

In general, free-floating organisms may be vulnerable to antibiotics. However, bacteria and fungi may become impervious to antibiotics by attaching to surfaces and producing a slimy protective substance, often referred to as extra-cellular polymeric substance (EPS), polysaccharide covering or glycocalyx. As the microbes proliferate, more than 50 genetic up or down regulations may occur, resulting in the formation of a more antibiotic resistant microbial biofilm. One article attributes two-thirds of the bacterial infections that physicians encounter to biofilms. SCIENCE NEWS, 1-5 Jul. 14, 2001.

Biofilm formation is a genetically controlled process in the life cycle of bacteria that produces numerous changes in the cellular physiology of the organism, often including increased antibiotic resistance (of up to 100 to 1000 times), as compared to growth under planktonic (free floating) conditions. As the organisms grow, problems with overcrowding and diminishing nutrition trigger shedding of the organisms to seek new locations and resources. The newly shed organisms quickly revert back to their original free-floating phase and are once again vulnerable to antibiotics. However, the free-floating organism may enter the bloodstream of the patient, creating bloodstream infections, which produce clinical signs, e.g. fever, and more serious infection-related symptoms. Sessile rafts of biofilm may slough off and may attach to tissue surfaces, such heart valves, causing proliferation of biofilm and serious problems, such as endocarditis.

To further complicate matters, conventional sensitivity tests measure only the antibiotic sensitivity of the free-floating organisms, rather than organisms in a biofilm state. As a result, a dose of antibiotics is administered to the patient, such as through a catheter, in amounts that rarely have the desired effect on the biofilm phase organisms that may reside in the catheter. The biofilm organisms may continue to shed more planktonic organisms or may go dormant and proliferate later as an apparent recurrent infection.

In order to eradicate biofilm organisms through the use of antibiotics, a laboratory must determine the concentration of antibiotic required to kill the specific genetic biofilm phase of the organism. Highly specialized equipment is required to provide the minimum biofilm eradication concentration. Moreover, the current diagnostic protocols are time consuming, and results are often not available for many days, e.g. five (5) days. This time period clearly doesn't allow for prompt treatment of infections. The delay and the well-justified fear of infection may result in the overuse of broad-spectrum antibiotics and continued unnecessary catheter removal and replacement procedures. Overuse of broad-spectrum antibiotics can result in the development of antibiotic resistant bacterial strains, which cannot be effectively treated. Unnecessary catheter removal and replacement is painful, costly and may result in trauma and damage to the tissue at the catheter insertion site.

The antibiotic resistance of biofilms, coupled with complications of antibiotic use, such as the risk of antibiotic resistant strains developing, has made antibiotic treatment an unattractive option. As a result, antibiotic use is limited to symptomatic infections and prophylactic antibiotics are not typically applied to prevent contamination. Because the biofilm can act as a selective phenotypic resistance barrier to most antibiotics, the catheter must often be removed in order to eradicate a catheter related infection. Removal and replacement of the catheter is time consuming, stressful to the patient, and complicates the medical procedure. Therefore, there are attempts to provide convenient and effective methods for killing organisms, and especially those dwelling inside of catheters, without the necessity of removing the catheter from the body.

In addition to bacterial and fungal infections, amoebic infections can be very serious and painful, as well as potentially life threatening. Several species of Acanthamoeba, for example, have been found to infect humans. Acanthamoeba are found worldwide in soil and dust, and in fresh water sources as well as in brackish water and sea water. They are frequently found in heating, venting and air conditioner units, humidifiers, dialysis units, and in contact lens paraphernalia. Acanthamoeba infections, in addition to microbial and fungal infections, may also be common in connection with other medical and dental devices, including toothbrushes, dentures and other dental appliances, and the like. Acanthamoeba infections often result from improper storage, handling and disinfection of contact lenses and other medical devices that come into contact with the human body, where they may enter the skin through a cut, wound, the nostrils, the eye, and the like.

There is a need for improved methods and substances to prevent and destroy infections in catheters. Such disinfectant solutions should have a broad range of antimicrobial properties. In particular, the solutions should be capable of penetrating biofilms to eradicate the organisms comprising the biofilms. The methods and solutions should be safe enough to be use as a preventive measure as well as in the treatment of existing infections.

Poly(hexamethylenebiguanide) (PHMB) is a broad spectrum, fast acting disinfectant. It is used as a preservative for make-up removers, moisturizing toners, facial cleansers, wet wipes and offers antibacterial and deodorant properties. It is available as Poly(hexamethylenebiguanide) hydrochloride (commonly known as polihexanide) in a solution form at a concentration of 20%. It is sold under the name of Cosmocil CQ via Avecia/Arch Chemicals.

Ethylene diamine tetraacetic acid (EDTA) has been used for systemic detoxification treatment and as an anticoagulant in blood samples for some time. Thus its use for medical treatment and applications is established. The use of disodium EDTA and calcium disodium EDTA in combination with other compounds to enhance anti-microbial properties of these other compounds has been studied and practiced. It has been discovered that many stand-alone salts of Ethylenediamine-tetraaceticacid (EDTA) are effective anti-microbial agents and that specific salts are more effective than others. In particular, it has been discovered that certain salts of EDTA exhibit anti-microbial (both antifungal and antibacterial) properties superior to those of the disodium salt in common use. In particular, dipotassium and ammonium EDTA are superior to disodium EDTA, and tetrasodium EDTA (TEDTA) has been found to be preferred to disodium, ammonium, and dipotassium.

### OBJECTS AND SUMMARY

In the following discussion, the terms “microbe” or “microbial” will be used to refer to microscopic organisms or matter, including fungal and bacterial organisms, and possibly including viral organisms, capable of infecting humans. The term “anti-microbial” will thus be used herein to refer to a material or agent that kills or otherwise inhibits the growth of fungal and/or bacterial and possibly viral organisms.

The term “disinfect” will be used to refer to the reduction, inhibition, or elimination of infectious microbes from a defined system. The term “disinfectant” will be used herein to refer to a one or more anti-microbial substances used either alone or in combination with other materials such as carriers, solvents, or the like.

The term “bactericidal activity” is used to refer to an activity that at least essentially kills an entire population of bacteria, instead of simply just reducing or inhibiting their growth. The term “fungicidal activity” is used to refer to an activity that at least essentially kills an entire population of yeast, instead of simply just reducing or inhibiting their growth. Contamination of conduits, e.g., catheters, poses serious and substantial health risks and bactericidal disinfection is a significant priority.

The term “infected system” will be used herein to refer to a defined or discrete system or environment in which one or more infectious microbes are or are likely to be present. Examples of infected systems include a physical space such as a bathroom facility or operating room, a physical object such as food or surgical tool, a biological system such as the human body, or a combination of a physical object and a biological system such as a catheter or the like arranged at least partly within a human body. Tubes and other conduits for the delivery of fluids, in industrial and healthcare settings, may also define an infected system.

A solution that consists essentially of PHMB and EDTA salt(s) in a solvent, such as water or saline, is substantially free from other active substances having antimicrobial and/or anti-fungal activity.

The present disclosure involves disinfectant solutions comprising, or consisting essentially of, or consisting of, PHMB and EDTA salt(s) at a prescribed concentration and/or pH. The inventors have discovered, unexpectedly, that certain PHMB and EDTA salt(s) formulations provide enhanced disinfectant activities. PHMB and EDTA salt(s) formulations act as enhanced, fast acting catheter lock/flush solutions. PHMB and EDTA salt(s) formulations of the present disclosure are also highly effective in killing pathogenic biofilm organisms, and are expected to be effective in reducing existing biofilms, in eliminating existing biofilms as well as preventing biofilm formation. PHMB and EDTA salt(s) formulations function as broad-spectrum anti-microbial agents, as well as fungicidal agents against many strains of pathogenic yeast. PHMB and EDTA salt(s) formulations are expected to exhibit anti-protozoan activity and also exhibit anti-amoebic activity.

The PHMB and EDTA salt(s) formulations of the present disclosure are safe for human administration and are biocompatible and non-corrosive. The disinfectant solutions of the present disclosure have applications at least as lock and lock flush solutions for various types of catheters. The efficacy of the PHMB and EDTA salt(s) formulations of the present disclosure is superior to many disinfectant compositions conventionally used as catheter lock/flush solutions. The disclosed PHMB and EDTA salt(s) formulations do not contribute to antibiotic resistance, which provides yet another important benefit.

The PHMB and EDTA salt(s) formulations of the present disclosure are also have improved anticoagulant properties and are thus especially beneficial as catheter lock-flush solutions and other related uses.

In one embodiment, disinfectant compositions of the present disclosure have some of the following properties: anticoagulant properties; inhibitory and/or bactericidal activity against a broad spectrum of bacteria in a planktonic form; inhibitory and/or fungicidal activity against a spectrum of fungal pathogens; inhibitory and/or bactericidal activity against a broad spectrum of bacteria in a sessile form; inhibitory activity against protozoan infections; inhibitory activity against Acanthamoeba infections; safe and biocompatible, at least in modest volumes, in contact with a patient; and safe and biocompatible, at least in modest volumes, in a patient's bloodstream.

Methods for inhibiting the growth and proliferation of microbial populations and/or fungal pathogens are provided that comprise contacting an infected or suspected infected object, or surface, e.g., catheter, with a disinfectant composition of the present disclosure. Methods for inhibiting the growth and proliferation of protozoan populations are also provided, comprising contacting an infected or suspected infected object, or surface, e.g., catheter, with a disinfectant composition of the present disclosure.

Methods for inhibiting the growth and proliferation of amoebic populations, and for preventing amoebic infection, particularly Acanthamoeba infections, are provided, comprising contacting an object, or a surface, e.g., catheter, with a disinfectant composition of the present disclosure. Methods for substantially eradicating microbial populations are also provided and comprise contacting an infected or suspected infected object, or surface, e.g., catheter, with a disinfectant composition of the present disclosure. Methods for substantially eradicating an Acanthamoeba population are provided and comprise contacting an infected or suspected infected object, or surface, e.g., catheter, with a disinfectant composition of the present disclosure. Depending on the disinfectant composition used in the various methods, various compositions and contact time periods may be required to inhibit the formation and proliferation of various populations, and/or to substantially eradicate various populations. Suitable contact time periods for various compositions may be determined by routine experimentation.

Importantly, in most embodiments, disinfectant compositions and methods of the present disclosure do not employ traditional antibiotic agents and thus do not contribute to the development of antibiotic resistant organisms.

In one embodiment, disinfectant compositions consisting of, consisting essentially of, or comprising PHMB and EDTA salt(s) at a greater than physiological pH are provided as disinfectant compositions of the present disclosure. Such disinfectant compositions have application as lock solutions and lock flush solutions for various types of in-dwelling access catheters, including vascular catheters used for delivery of fluids, blood products, drugs, nutrition, withdrawal of fluids or blood, dialysis, monitoring of patient conditions, and the like. Disinfectant solutions of the present disclosure may also be used as lock and lock flush solutions for urinary catheters, nasal tubes, throat tubes, and the like. The general solution parameters described below are suitable for these purposes. In one embodiment, a disinfectant solution consisting of, consisting essentially of, or comprising PHMB and EDTA salt(s) at a greater than physiological pH is provided to maintain the patency of in-dwelling intravascular access devices. Methods for disinfectant catheters and other medical tubes, such as nasal tubes, throat tubes, and the like, are also provided and involve contacting the catheter or other medical tube with a disinfectant composition of the present disclosure.

### BRIEF DESCRIPTION OF THE DRAWING(S)

FIG. 1 shows the results of experiments of a PHMB MIC test with P. aeruginosa. The data suggests that the MIC value for PHMB is <5 PPM.

FIG. 2 shows the results of experiments of a PHMB MIC test with S. aureus. The data suggests that the MIC value for PHMB is <1.25 PPM.

FIG. 3 shows the results of experiments of a PHMB MIC test with C. Albicans. The data suggests that the MIC value for PHMB is <1.25 PPM.

FIG. 4 shows the results of experiments of a PHMB MBC test with C. Albicans. The data suggests that the MBC value for PHMB is <1.25 PPM.

FIG. 5 shows the results of experiments of a EDTA(Na₄) MIC test with P. aeruginosa. The data suggests that the MIC value for EDTA(Na₄) is <0.25 wt %.

FIG. 6 shows the results of experiments of a EDTA(Na₄) MIC test with S. aureus. The data suggests that the MIC value for EDTA(Na₄) is <0.03125 wt %.

FIG. 7 shows the results of experiments of a EDTA(Na₄) MIC test with C. Albicans. The data suggests that the MIC value for EDTA(Na₄) is <0.03125 wt %.

FIG. 8 shows the results of experiments of a EDTA(Na₄) MBC test with C. Albicans. The data suggests that the MBC value for EDTA(Na₄) is <0.0625 wt %.

FIG. 9 shows the results of experiments of a Checkerboard Titration with S. aureus. The data suggests that the FIC index=0.8 for EDTA(Na₄)+PHMB Combination.

FIG. 10 shows the results of experiments of a Checkerboard Titration with P. aeruginosa. The data suggests that the FIC index=0.5 for EDTA(Na₄)+PHMB Combination.

FIG. 11 shows the results of experiments of a Checkerboard Titration with C albicans. The data suggests that the FIC index=0.6 for EDTA(Na₄)+PHMB Combination.

FIG. 12 shows the results of experiments of a Rate Kill Assay for S. aureus. The data clearly suggest the synergistic action against S. Aureus by EDTA(Na₄)+PHMB combination.

FIG. 13 shows the results of experiments of a Rate Kill Assay for P. aeruginosa. The data clearly suggest the synergistic action against P. aeruginosa by EDTA(Na₄)+PHMB combination.

FIG. 14 shows the results of experiments of a Rate Kill Assay for C. albicans. The data does not suggest the synergistic action against C. albicans by EDTA(Na₄)+PHMB combination. However, the data suggests the combination is very effective against C. albicans with PHMB being the dominant component.

FIG. 15 shows the results of experiments of a PHMB MIC and MBC test with S. aureus at a pH of 7. The data suggests that the MIC value for PHMB at a pH of 7 is <5 PPM. The data suggests that the MBC value for PHMB at a pH of 7 is <5 PPM.

FIG. 16 shows the results of experiments of a PHMB MIC and MBC test with P. aeruginosa at a pH of 7. The data suggests that the MIC value for PHMB at a pH of 7 is <5 PPM. The data suggests that the MBC value for PHMB at a pH of 7 is <5 PPM.

FIG. 17 shows the results of experiments of a PHMB MIC and MBC test with C. albicans at a pH of 7. The data suggests that the MIC value for PHMB at a pH of 7 is <10 PPM. The data suggests that the MBC value for PHMB at a pH of 7 is <10 PPM.

FIG. 18 shows the results of experiments of a EDTA MIC and MBC test with S. aureus at a pH of 7. The data suggests that the MIC value for EDTA at a pH of 7 is <0.03 wt %. The data suggests that the MBC value for EDTA at a pH of 7 is <0.13 wt %.

FIG. 19 shows the results of experiments of a EDTA MIC and MBC test with P. aeruginosa at a pH of 7. The data suggests that the MIC value for EDTA at a pH of 7 is <0.25 wt %. The data suggests that the MBC value for EDTA at a pH of 7 is <4.00 wt %.

FIG. 20 shows the results of experiments of a EDTA MIC test with C. albicans at a pH of 7. The data suggests that the MIC value for EDTA at a pH of 7 is >4.0 wt %. The MBC value for EDTA at a pH of 7 could not be determined.

FIG. 21 shows the results of experiments of a Checkerboard Titration with S. aureus at a pH of 7. The data suggests that the FIC index=0.6 for PHMB-EDTA Combination at a pH of 7.

FIG. 22 shows the results of experiments of a Checkerboard Titration with P. aeruginosa at a pH of 7. The data suggests that the FIC index=0.5 for PHMB-EDTA Combination at a pH of 7.

FIG. 23 shows the results of experiments of a Checkerboard Titration with C. albicans at a pH of 7. The data suggests that there is no synergy against C. ablicans for PHMB-EDTA Combination at a pH of 7.

FIG. 24 shows the results of experiments (raw data) of a Prothrombin Time (PT) Assay.

FIG. 25 shows the results of experiments (processed data) of a Prothrombin Time (PT) Assay.

FIG. 26 shows the graph of the International Normalized Ratio (INR) for EDTA(Na₄) from a Prothrombin Time (PT) Assay.

FIG. 27 shows the graph of the International Normalized Ratio (INR) for PHMB from a Prothrombin Time (PT) Assay.

FIG. 28 shows the graph of the International Normalized Ratio (INR) for combined EDTA(Na₄) and PHMB formulations from a Prothrombin Time (PT) Assay.

### DETAILED DESCRIPTION

Disinfectant compositions of the present disclosure may comprise concentrations of PHMB and EDTA salt(s) at a pH higher than physiological. PHMB and EDTA salt(s) may be used in compositions with water as the solvent.

Some properties of PHMB are:

Physical Properties
Color—Colorless to slightly pale yellow
Solubility—Miscible with water, ethanol, glycerine and propylene glycol
Specific Gravity at 25° C.-1.04
pH—5.0-5.5
Shelf Life—greater than two year storage stability
Stability—Effective and stable over a broad pH range (4-10) active agent
heat stable to >140° C.
UV stable
odorless, non-foaming
Chemically stable and non-volatile

Chemical Properties
Zero Volatile Organic Compound
Compatible with a wide range of cosmetic raw materials
Compatible with cationic, amphoteric and non-ionic surfactants
Incompatible with strongly anionic systems

Antimicrobial Properties
Unique biguanide chemistry
Novel non-specific mode of action
No known evidence of development of organism resistance
Contains no formaldehyde and is not a formaldehyde donor
Broad spectrum of activity high activity vs. tough Gram (negative) organisms, e.g., Pseudomonas
Extensively studied mammalian toxicity
Low acute toxicity via dermal and oral routes
Low skin and eye irritancy potential at in-use concentration
Slow toxicity following long term exposure
Not teratogenic and shows no reproductive effects when studied over two generations
Non-genotoxic in a range of studies
Not considered carcinogenic in humans.

Compositions comprising PHMB have a well established safety profile in connection with medical usage and administration to humans. Acute Oral LD₅₀ of 1617 mg/kg (see table below for further info).

| | | | | |
|---|---|---|---|---|
| Guideline | | | | Toxicity |
| No. | Study Type | MRID #(s) | Results | Category |

Acute Toxicity

| | | | | |
|---|---|---|---|---|
| 870.1100 | Acute Oral | 00030330 | LD50 = 2747 mg/kg | III |
| | | 44940701 | LD50 = 1831 mg/kg (M) LD50 = | |
| | | | 1617 mg/kg (F) | |
| | | 45916505 | LD50 = 1049 mg/kg (F) | |
| 870.1200 | Acute Dermal | 00065124 | LD50 > 2.0 ml/kg | III |
| | | 44940702 | LD50 > 2000 mg/kg | |
| | | 45916506 | LD50 > 5000 mg/kg | IV |
| 870.1300 | Acute Inhalation | 44970403 | LC50 = 1.76 mg/L | III |
| 870.2400 | Primary Eye Irritation | 00046789 | Moderate irritant | II |
| | | 00065120 | | |
| | | 44963902 | | |
| 870.2500 | Primary Skin Irritation | 00046789 | Moderate irritant | II |
| | | 00065120 | | |
| | | 44949704 | Slight irritant | IV |
| | | 45916509 | | |
| 870.2600 | Dermal Sensitization | 42674201 | Moderate sensitizer | NA |
| | | 44940705 | Mild sensitizer | |

Notes:
LC = Lethal Concentration;
LD = Lethal Dose;
NA = Not Applicable

| | | Special FQPA SF* | |
|---|---|---|---|
| | | and Level of | |
| Exposure | Dose Used in Risk | Concern for Risk | |
| Scenario | Assessment, UF | Assessment | Study and Toxicological Effects |
| Acute Dietary | NOAEL = 20 mg/kg/day | FQPA SF = 1 | Rabbit Developmental Study |
| (Females 13-50 | UF = 100 | aPAD = acute RfD | (MRID 42865901) |
| years of age) | Acute RfD = 0.2 mg/kg/day | FQPA SF = | LOAEL = 40 mg/kg/day based on reduced |
| | | 0.2 mg/kg/day | number of litters and skeletal |
| | | | abnormalities. |
| Acute Dietary | No Appropriate single dose effects can be selected for general population | | |
| (General population | | | |
| including infants and | | | |
| children) | | | |
| Chronic Dietary (All | NOAEL = 20 mg/kg/day | FQPA SF = 1 cPAD = | Rabbit Developmental Study (MRID |
| populations) | UF = 100 | chronic RfD FQPA | 42865901) LOAEL = 40 mg/kg/day |
| | Chronic RfD = 0.2 mg/kg/day | SF = 0.2 mg/kg/day | Based on the increased mortality, reduced |
| | | | food consumption, and clinical toxicity; |
| | | | Mouse Developmental Study (Report No. |
| | | | CTL/P/335, 1977 (cited in Report No. |
| | | | 003810, 1978. Section C-9)) |
| | | | LOAEL = 40 mg/kg/day; |
| | | | Based on reduced body weight gain; and |
| | | | Rat Developmental Study (Report No. |
| | | | CTL/P/1262, 1976 (cited in Report No. |
| | | | 003810, 1978. Section C-11)) |
| | | | LOAEL = 50 mg/kg/day |
| | | | Based on reduced food consumption. |

| | |
|---|---|
| Cancer (Oral, | The HED Cancer Assessment Review Committee (CARC) classified PHMB as |
| dermal, Inhalation) | “Suggestive Evidence of Carcinogenicity, but not sufficient to Assess Human Carcinogenic |
| | Potential” by the oral and dermal routes. Quantification of human cancer risk is not |
| | required. |

Notes:
UF = uncertainty factor,
FQPA SF = Food Quality Protection Act safety factor,
NOAEL = no observed adverse effect level,
LOAEL = lowest observed adverse effect level,
PAD = population adjusted dose (a = acute, c = chronic)
RfD = reference dose
Reference —Re-registration Eligibility Decision for PHMB, September 2005.

PHMB is also present, in combination with other components, in many solutions used in medical and human health applications, and has been established as safe for human use, both in vitro and in vivo. PHMB is readily available at a reasonable cost, and is stable over time in solution.

Soluble salts of EDTA are used in compositions of the present disclosure. Sodium salts of EDTA are commonly available and generally used, including di-sodium, tri-sodium and tetra-sodium salts, although other EDTA salts, including ammonium, di-ammonium, potassium, di-potassium, cupric di-sodium, magnesium di-sodium, ferric sodium, and combinations thereof, may be used, provided they have the antibacterial and/or fungicidal and/or anti-protozoan and/or anti-amoebic properties desired, and provided that they are sufficiently soluble in the solvent desired. Various combinations of EDTA salts may be used and may be preferred for particular applications.

The British Pharmacopoeia (BP) specifies that a 5% solution of di-sodium EDTA has a pH of 4.0 to 5.5. The BP also specifies a pH range of 7.0 to 8.0 for solutions of tri-sodium EDTA. At physiological pH, the sodium salts of EDTA exist as a combination of di-sodium and tri-sodium EDTA, with the tri-sodium salt of EDTA being predominant. In the U.S., pharmaceutical “di-sodium” EDTA prepared for injection has generally been titrated with sodium hydroxide to a pH of 6.5 to 7.5. At this pH, the EDTA solution actually comprises primarily tri-sodium EDTA, with a lesser proportion of the di-sodium salt. Other compositions comprising sodium salts of EDTA that are used in medical or healthcare applications are generally adjusted to a pH that is substantially physiological.

Compositions comprising EDTA have a well established safety profile in connection with medical usage and administration to humans. Doses of up to 3000 mg EDTA disodium are infused over 3 hours, on a daily basis, for the treatment of hypercalcemia in humans. This dose is well tolerated. EDTA salts are also present, in combination with other components, in many solutions used in medical and human health applications, and have been established as safe for human use, both in vitro and in vivo. EDTA salts are readily available at a reasonable cost, and are stable over time in solution.

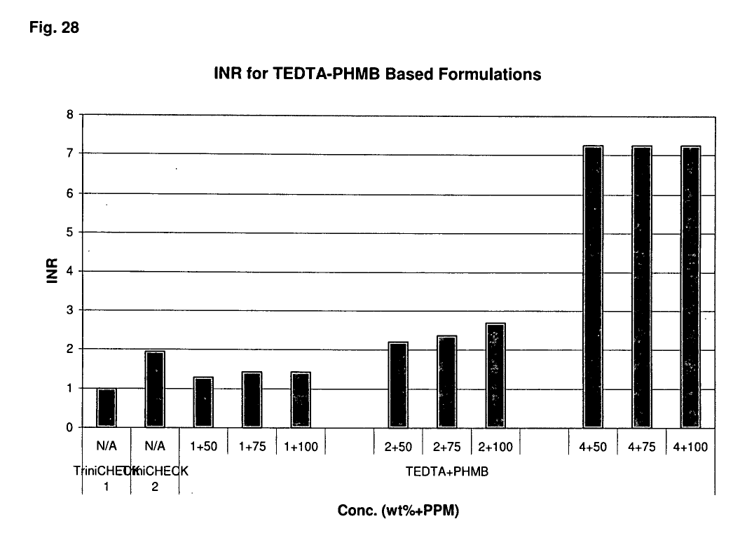

The combination of PHMB and EDTA salt(s) has an anti-coagulant effect. The anti-coagulant effect is further detailed in FIG. 28.

Embodiments of the disclosed composition may comprise at least 0.1 PPM PHMB and up to 400 PPM PHMB. Embodiments comprising at least 5 PPM PHMB and less than 200 PPM PHMB are preferred for many applications, and compositions comprising about 10-50 PPM PHMB are especially preferred.

Embodiments of the disclosed composition may comprise at least 0.0125% EDTA salt(s), by weight per volume solution (w/v) and up to 12.0% (w/v) EDTA salt(s). Embodiments comprising at least 0.25% (w/v) EDTA salt(s) and less than 8% (w/v) EDTA salt(s) are preferred for many applications, and compositions comprising about 0.5-4 (w/v) EDTA salt(s) are especially preferred.

Embodiments of the disclosed composition may comprise between 0 and 25% (v/v) ethanol and water. Other embodiments of the disclosed composition may comprise between 0 and 20% (v/v) ethanol and water, between 0 and 15% (v/v) ethanol and water, or between 0 and 10% (v/v) ethanol and water.

The desired PHMB and EDTA salt(s) concentrations for various applications may depend on the type of infection being treated and, to some degree, on the solvent used for disinfectant compositions. When aqueous solvents comprising ethanol are used, for example, the concentrations of PHMB and EDTA salt(s) required to provide the desired level of activity may be reduced compared to the PHMB and EDTA salt(s) concentrations used in compositions having water as the solvent. “Effective” concentrations of PHMB and EDTA salt(s) in disinfectant compositions of the present disclosure for inhibitory, bactericidal, fungicidal, biofilm eradication and other purposes, may be determined by routine experimentation.

In certain embodiments, disinfectant compositions of the present disclosure comprise, or consist essentially of, or consist of, PHMB and EDTA salt(s) in solution at a pH higher than physiological, preferably at a pH of > or >8.0, or at a pH > or >8.5, or at a pH> or >9, or at a pH> or >9.5, or at a pH>or >10.0, or at a pH> or >10.5. Compositions comprising PHMB and EDTA salt(s) that are used in medical or healthcare applications may be adjusted to a pH that is substantially physiological. In one embodiment, disinfectant compositions of the present disclosure comprise, or consist essentially of, or consist of, PHMB and a sodium EDTA salt (or combination of sodium salts) in solution at a pH in the range between 8.5 and 12.5 and, in another embodiment, at a pH of between 9.5 and 11.5 and, in yet another embodiment, at a pH of between 10.5 and 11.5. When used herein, the term “EDTA salt” may refer to a single salt, such as a di-sodium or tri-sodium or tetra-sodium salt, or another EDTA salt form, or it may refer to a combination of such salts. The composition of EDTA salt(s) depends both on the EDTA salts used to formulate the composition, and on the pH of the composition. For disinfectant compositions of the present disclosure comprising sodium EDTA salt(s), and at the desired pH ranges (specified above), the sodium EDTA salts are predominantly present in both the tri-sodium and tetra-sodium salt forms.

Disinfectant compositions comprising, or consisting essentially of, or consisting of PHMB and EDTA salt(s) have different “effective” pH ranges. “Effective” pH ranges for desired EDTA salt(s) in disinfectant compositions of the present disclosure for inhibitory, bactericidal, fungicidal, biofilm eradication and other purposes, may be determined by routine experimentation.

In some embodiments, disinfectant compositions of the present disclosure consist of PHMB and EDTA salt(s), as described above, and disinfectant solutions consist of PHMB and EDTA salt(s) dissolved in a solvent, generally an aqueous solvent such as water or saline. In other embodiments, disinfectant compositions of the present disclosure consist essentially of PHMB and EDTA salt(s), as described above, generally in an aqueous solvent such as water or saline.

In some embodiments, disinfectant compositions of the present disclosure comprise PHMB and EDTA salt(s) having specified concentrations, at specified pH ranges, and may contain materials, including active components, in addition to the PHMB and EDTA salt(s) described above. Other antimicrobial or biocidal components may be incorporated in disinfectant compositions of the present disclosure comprising PHMB and EDTA salt(s), although the use of traditional antibiotics and biocidal agents is generally discouraged as a result of the potential dire consequences of the development of antibiotic- and biocidal-resistant organisms. In some embodiments, disinfectant compositions of the present disclosure comprising PHMB and EDTA salt(s) having specified concentration(s), at specified pH ranges, are substantially free from other active substances having substantial antimicrobial and/or anti-fungal activity.

Other active and inactive components may also be incorporated in disinfectant compositions of the present disclosure comprising PHMB and EDTA salt(s), preferably provided that they don't deleteriously affect the activity and/or stability of the PHMB and EDTA salt(s). Proteolytic agents may be incorporated in disinfectant compositions for some applications. Disinfectant compositions formulated for topical application have various creams, emollients, skin care compositions such as aloe vera, and the like, for example. Disinfectant compositions of the present disclosure provided in a solution formulation may also comprise other active and inactive components, preferably provided they don't interfere, deleteriously, with the activity and/or stability of the PHMB and EDTA salt(s).

The compositions of the present disclosure may be used in a solution or a dry form. In solution, the PHMB and EDTA salt(s) are preferably dissolved in a solvent, which may comprise an aqueous solution, such as water or saline, or another biocompatible solution in which the PHMB and EDTA salt(s) are soluble. Other solvents, including alcohol solutions, may also be used. In one embodiment, PHMB and EDTA salt(s) compositions of the present disclosure may be formulated in a mixture of water and ethanol. Such solutions are expected to be highly efficacious and may be prepared by making a concentrated PHMB and EDTA salt(s) stock solution in water and then introducing the desired concentration of ethanol. Ethanol concentrations of from more than about 0.5% and less than about 10%, v/v, are expected to provide effective disinfectant compositions. In some embodiments, bio-compatible non-aqueous solvents may also be employed, provided the EDTA salt(s) can be solubilized and remain in solution during storage and use.

PHMB and EDTA salt(s) solutions of the present disclosure are preferably provided in a sterile and non-pyrogenic form and may be packaged in any convenient fashion. In some embodiments, disinfectant PHMB and EDTA salt(s) compositions of the present disclosure may be provided in connection with or as part of a medical device, such as in a pre-filled syringe or another medical device. The compositions may be prepared under sterile, aseptic conditions, or they may be sterilized following preparation and/or packaging using any of a variety of suitable sterilization techniques. Single use vials, syringes or containers of PHMB and EDTA salt(s) solutions may be provided. Multiple use vials, syringes or containers may also be provided. Systems of the present disclosure include such vials, syringes or containers containing the PHMB and EDTA salt(s) solutions of the present disclosure. Catheters contemplated for use include peripherally inserted catheters, central venous catheters, peritoneal catheters, hemodialysis catheters and urological catheters.

The compositions of the present disclosure may also be provided in a substantially “dry” form, such as a substantially dry coating on a surface of tubing, or a conduit, or a medical device such as a catheter or conduit, or a container, or the like. Dry forms of the disinfectant compositions of the present disclosure may include hydrophilic polymers such as PVP, which tend absorb water and provide lubricity, surfactants to enhance solubility and/or bulking and buffering agents to provide thermal as well as pH stability. Such substantially dry forms of PHMB and EDTA salt(s) compositions of the present disclosure may be provided in a powder or lyophilized form that may be reconstituted to form a solution with the addition of a solvent. Substantially dry forms of PHMB and EDTA salt(s) compositions may alternatively be provided as a coating, or may be incorporated in a gel or another type of carrier, or encapsulated or otherwise packaged and provided on a surface as a coating or in a container. Such substantially dry forms of PHMB and EDTA salt(s) compositions of the present disclosure are formulated such that in the presence of a solution, the substantially dry composition forms an PHMB and EDTA salt(s) solution having the composition and properties described above. In certain embodiments, different encapsulation or storage techniques may be employed such that effective time release of the PHMB and EDTA salt(s) is accomplished upon extended exposure to solutions. In this embodiment, the substantially dry PHMB and EDTA salt(s) solutions may provide disinfectant activity over an extended period of time and/or upon multiple exposures to solutions.

Formulation and production of disinfectant compositions of the present disclosure are generally straightforward. In one embodiment, desired disinfectant compositions of the present disclosure are formulated by dissolving PHMB and EDTA salt(s) in an aqueous solvent, such as purified water, to the desired concentration and adjusting the pH of the solution to the desired pH. In alternative embodiments, desired disinfectant compositions of the present disclosure are formulated by dissolving PHMB and EDTA salt(s) in a solvent in which the PHMB and EDTA salt(s) are soluble to provide a concentrated, solubilized solution, and additional solvents or components may then be added, or the solubilized composition may be formulated in a form other than a solution, such as a topical preparation. The disinfectant solution may then be sterilized using conventional means, such as filtration and/or ultrafiltration, and other means. The preferred osmolarity range for PHMB and EDTA salt(s) solutions is from 240-500 mOsm/Kg, more preferably from 300-420 mOsm/Kg. The solutions are preferably formulated using USP materials.

A PHMB and EDTA salt(s) solution can be used as a treatment for catheters defining an infected system. The PHMB and EDTA salt(s) solution may inhibit microbe colonization by treating the catheter with the solution at the prescribed concentration using a liquid lock prior to and in between infusions and/or by surface coating of catheter devices. A further application is the treatment of colonized or infected catheters by use of a liquid lock containing the PHMB and EDTA salt(s) solution in the preferred concentration and pH.

Typically, the PHMB and EDTA salt(s) solution, when used to treat catheters, are dissolved in water as a carrier, although other carriers may be used. Substances such as thrombolytics, sodium, alcohol, or reagents may also be added to the basic water/PHMB and EDTA salt(s) solution.

### Minimum Inhibitory Concentration (MIC) Experiments

The minimum concentration of a composition required to inhibit growth is known as the minimum inhibitory concentration (MIC). In order to determine MIC and MBC (minimum bactericidal concentration) a National Committee on Clinical Laboratory Standards (NCCLS) micro-dilution procedure was followed. According to the procedure each formulation must be exposed to 6 log concentration (or the highest achievable concentration) of organism. In the current protocol 100 μL of MHB was mixed with 90 μL of formulation and 10 μL of log 8 concentration organism (or the highest achievable concentration). The concentration of the formulation was adjusted to obtain the required concentration in the final solution. The mixture was incubated at 37 degree C for 16-24 hrs. After 16-24 hours the absorbance value was read at 600 nm. The obtained data was corrected by subtracting the appropriate blanks. Finally, the wells having an absorbance >0.1 were marked + and <0.1 were marked −. The +symbol indicated growth while −symbol indicates no growth. The positive growth controls must have a corrective absorbance value of >0.5 and negative controls must have a corrected absorbance value of <0.1. In cases where the positive growth controls corrected absorbance is lower than 0.5, an alternate rule is utilized which is “absorbance <than 20% of positive growth control is marked as −growth, while absorbance >than 20% of positive growth control is marked as +growth”.

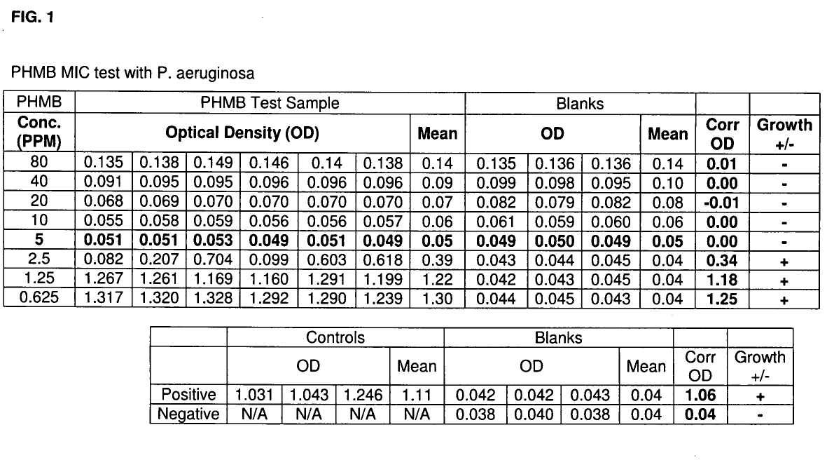

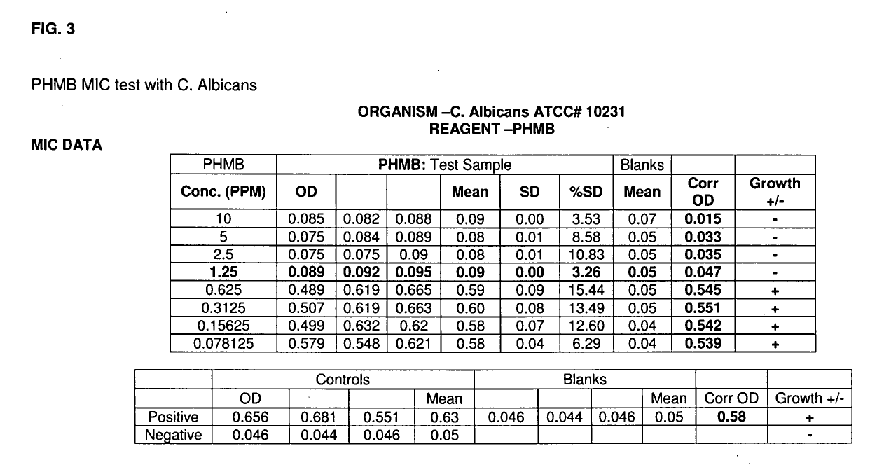

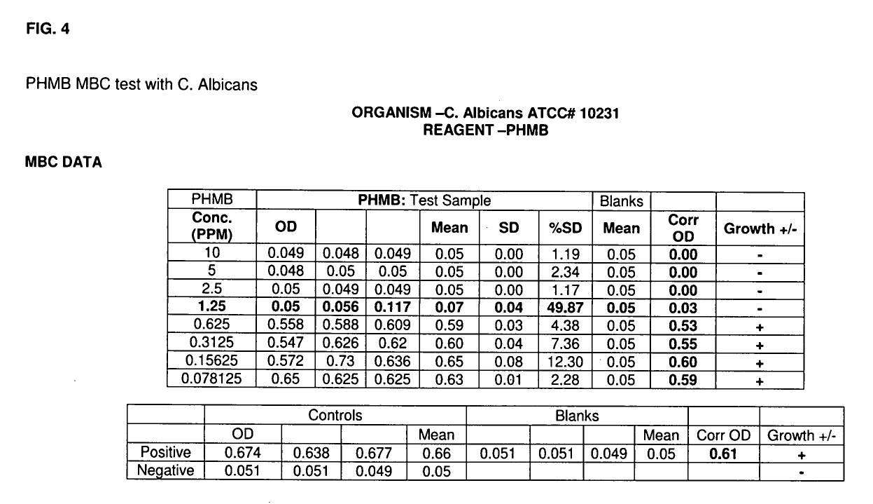

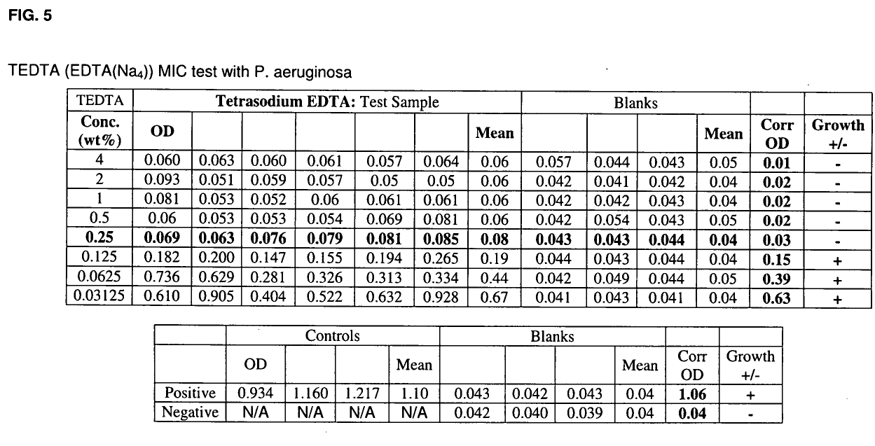

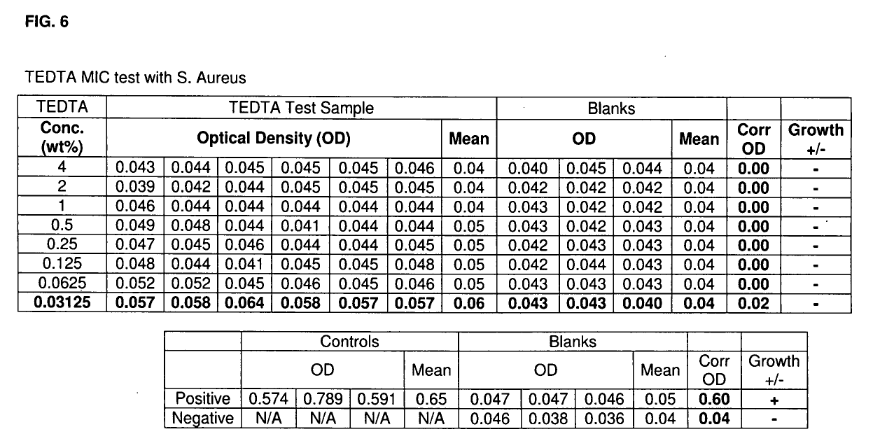

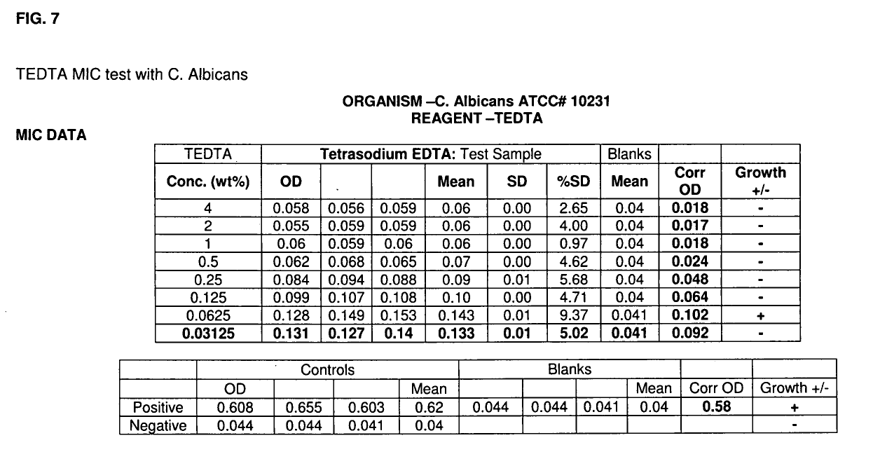

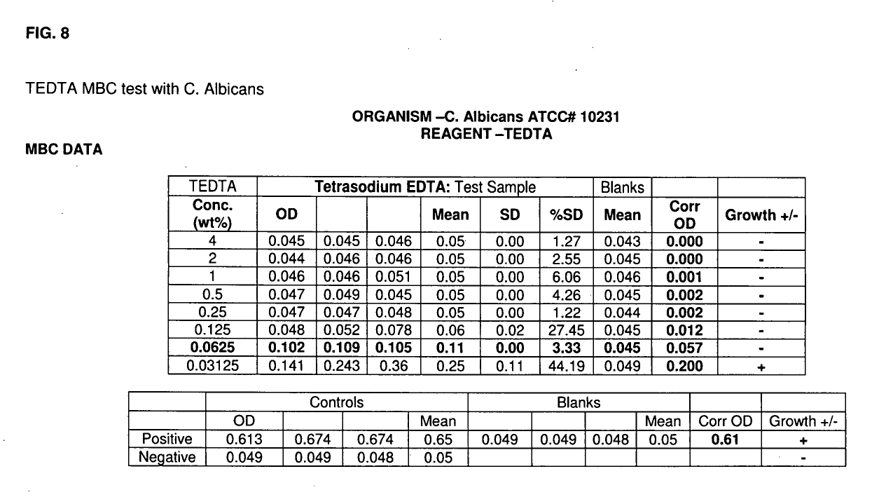

Staphylococcus aureus (Organism #25923), Pseudomonas aeruginosa (Organism #27853), and Candida Albicans (Organism #10231) was obtained from ATCC. PHMB was used (Avecia, Lot #1L15-038). EDTA, tetrasodium salt hydrate, was used (Alfa Aesar, Catalogue #A17385, Lot #J9570A). A 200 PPM PHMB solution in water was prepared. A 8 wt % EDTA(Na₄) solution in water was prepared. These solutions were then diluted as necessary to obtain the required concentrations. A minimum concentration of EDTA(Na₄) and PHMB that inhibited the growth of Staphylococcus aureus and P. aeruginosa was found. As per experiments conducted, EDTA(Na₄) has a MIC of <0.03% (w/v) for S. aureus, PHMB has a MIC of <1.25 PPM for S. aureus, EDTA(Na₄) has a MIC of <0.25% (w/v) for P. aeruginosa, PHMB has a MIC of <5 PPM for P. aeruginosa, EDTA(Na₄) has a MIC of <0.03125% (w/v) for C. albicans, PHMB has a MIC of <1.25 PPM for C. albicans, EDTA(Na₄) has a MBC of <0.0625% (w/v) for C. albicans, PHMB has a MBC of <1.25 PPM for C. albicans. See FIGS. 1-8 for MIC and MBC results.

### Synergism Experiment

Two sets of experiments were conducted to show an unexpected synergism of the disinfectant activity of a composition that includes both EDTA(Na₄) and PHMB.

The first experiment conducted was a screening experiment using checkerboard titration to assess if the combinations fall within a range having an FIC index value of <1. The method used was a NCCLS micro-dilution procedure

The second experiment conducted was a “rate of kill” assay. A rate of kill assay can confirm whether combinations are synergistic or not. In this assay the formulations are first exposed to organisms for a desired time (the current formulations readings were taken at 0, 1, 2, 3 and 24 hrs). Then a sample of the organisms and formulation mixture is serially diluted and plated to assess the log recovery. The organisms are allowed to grow and are checked for growth/log recovery after 24 hrs. The log recovery values obtained for individual components were compared with the combinations. Any combinations having >2 log reduction when compared with the most active compound used in the combination at any time point tested were labeled as synergistic (Comparison of methods for assessing synergic antibiotic interactions, International journal of antimicrobial agents, 15 (2000) 125-129).

According to the first and second experiments described above, experiments were conducted to investigate the effect of PHMB on the antimicrobial activity of EDTA(Na₄). PHMB was used (Avecia, Lot #1L15-038). EDTA, tetrasodium salt hydrate, was used (Alfa Aesar, Catalogue #A17385, Lot #J9570A).

### Checkerboard Titration Experiment—S. Aureus

The Checkerboard Titration method was used to assess the interactions between EDTA(Na₄) and PHMB. The Checkerboard Titration method is a frequently used technique where, for example, each agent (EDTA(Na₄) and PHMB) was tested at multiple dilutions lower than the MIC. During this experiment, EDTA(Na₄) and PHMB were tested in the combinations to assess if the combinations have an FIC index of <1. The following concentrations were tested:

| | Concentration | Concentration PHMB |
|---|---|---|
| Combination | EDTA(Na₄) (wt %) | (PPM) |
| 0.5 MIC + 0.5 MIC | 0.0156 | 0.625 |
| 0.4 MIC + 0.4 MIC | 0.0125 | 0.5 |
| 0.35 MIC + 0.35 MIC | 0.01093 | 0.4375 |
| 0.3 MIC + 0.3 MIC | 0.0093 | 0.375 |
| 0.25 MIC + 0.25 MIC | 0.00781 | 0.3125 |
| 0.125 MIC + 0.125 MIC | 0.0039 | 0.15625 |

Fraction Inhibitory Concentration (FIC) is defined as the MIC of the compound in combination divided by the MIC of the compound alone. If the FIC index is <0.5, the combination is interpreted to be synergistic; <1 but >0.5—as partially synergistic; =1 as additive; >1 but <4 as indifferent; and ≧4 as antagonistic. In order to calculate the FIC index the following calculations are performed for compounds A and B:
FIC-A=(MIC of A in combination)/(MIC of A alone)
FIC-B=(MIC of B in combination)/(MIC of B alone)
FIC-combination=FIC-A+FIC-B

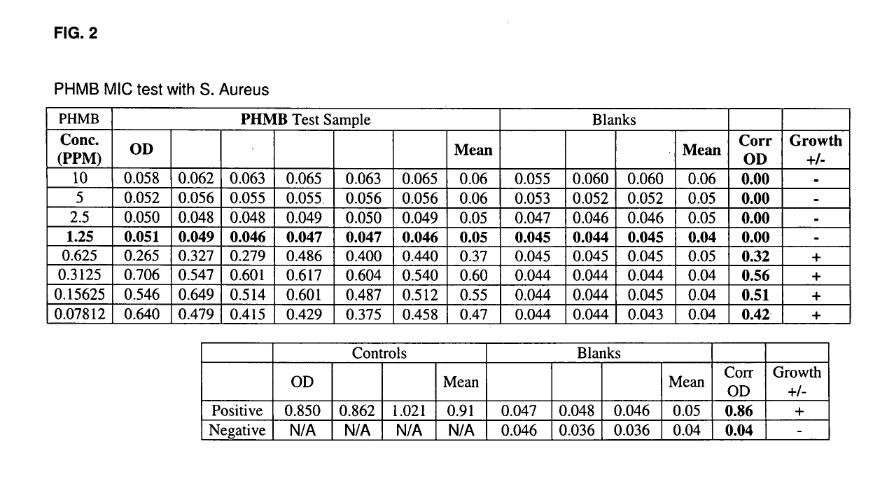

The MIC-PHMB (MIC of PHMB in combination with EDTA(Na₄)), a minimum concentration of PHMB, while in combination with EDTA(Na₄), that inhibited the growth of S. aureus in MHB was found. In order to determine the MIC-EDTA(Na₄) (MIC of EDTA(Na₄) in combination with PHMB ), a minimum concentration of EDTA(Na₄), while in combination with PHMB, that inhibited the growth of S. aureus in MHB was found. See FIG. 2, 6 and 9 for results.

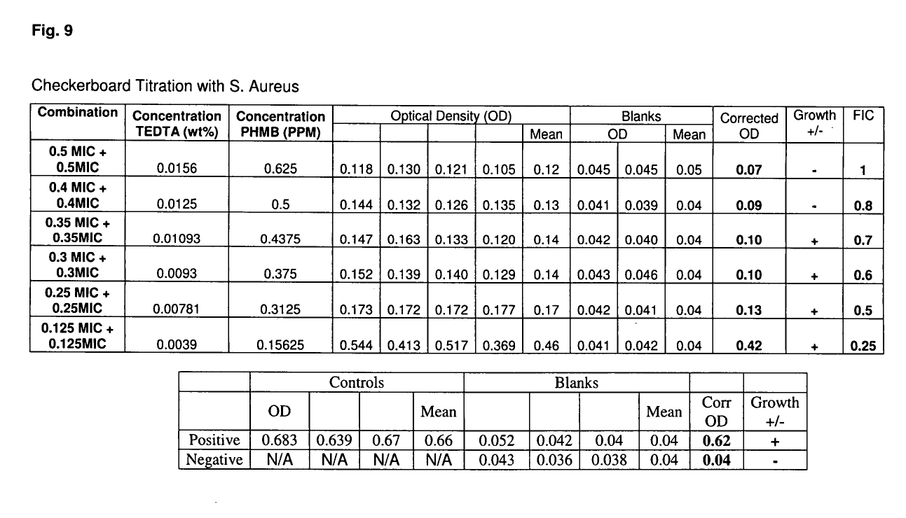

Thus, the FIC-PHMB is 0.4. The FIC-EDTA(Na₄) is 0.4. Thus, the FIC-combination is 0.4+0.4, which equals 0.80. See FIG. 9 for results. Accordingly, the combination of PHMB and EDTA(Na₄) unexpectedly has partial synergistic results. That is, embodiments of the combination of PHMB and EDTA(Na₄) provides results that are, unexpectedly, greater than the total effects of each agent operating by itself.

Checkerboard Titration Experiment—P. aeruginosa

The Checkerboard Titration method was used to assess the interactions between EDTA(Na₄) and PHMB. The Checkerboard Titration method is a frequently used technique where, for example, each agent (EDTA(Na₄) and PHMB) was tested at multiple dilutions lower than the MIC. During this experiment, EDTA(Na₄) and PHMB were tested in the combinations to assess if the combinations have an FIC index of <1. The following concentrations were tested:

| | Concentration | Concentration PHMB |
|---|---|---|
| Combination | EDTA(Na₄) (wt %) | (PPM) |
| 0.5 MIC + 0.5 MIC | 0.125 | 2.5 |
| 0.4 MIC + 0.4 MIC | 0.1 | 2 |
| 0.35 MIC + 0.35 MIC | 0.0875 | 1.75 |
| 0.3 MIC + 0.3 MIC | 0.075 | 1.5 |
| 0.25 MIC + 0.25 MIC | 0.0625 | 1.25 |
| 0.125 MIC + 0.125 MIC | 0.03125 | 0.625 |

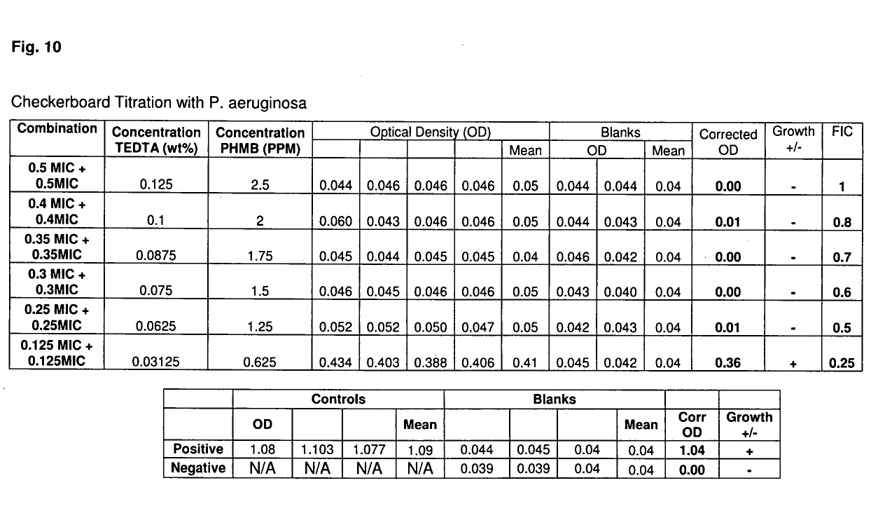

The FIC-PHMB is 0.25. The FIC-EDTA(Na₄) is 0.25. Thus, the FIC-combination is 0.25+0.25, which equals 0.5. See FIG. 10 for results. Accordingly, the combination of PHMB and EDTA(Na₄) unexpectedly has full synergistic results. That is, embodiments of the combination of PHMB and EDTA(Na₄) provides results that are, unexpectedly, greater than the total effects of each agent operating by itself.

Checkerboard Titration Experiment—C. albicans

The Checkerboard Titration method was used to assess the interactions between EDTA(Na₄) and PHMB. The Checkerboard Titration method is a frequently used technique where, for example, each agent (EDTA(Na₄) and PHMB) was tested at multiple dilutions lower than the MIC. During this experiment, EDTA(Na₄) and PHMB were tested in the combinations to assess if the combinations have an FIC index of <1. The following concentrations were tested:

| | Concentration | Concentration PHMB |
|---|---|---|
| Combination | EDTA(Na₄) (wt %) | (PPM) |
| 0.5 MIC + 0.5 MIC | 0.0156 | 0.625 |
| 0.4 MIC + 0.4 MIC | 0.0125 | 0.500 |
| 0.35 MIC + 0.35 MIC | 0.0109 | 0.438 |
| 0.3 MIC + 0.3 MIC | 0.0090 | 0.375 |
| 0.25 MIC + 0.25 MIC | 0.0078 | 0.313 |
| 0.125 MIC + 0.125 MIC | 0.0039 | 0.156 |

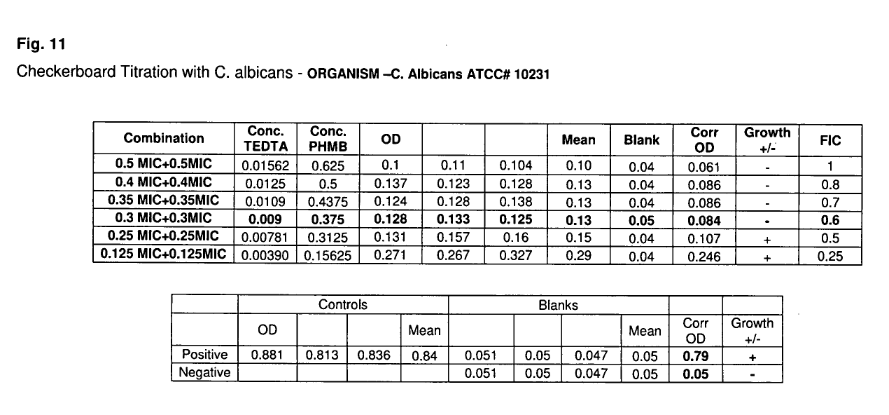

The FIC-PHMB is 0.3. The FIC-EDTA(Na₄) is 0.3. Thus, the FIC-combination is 0.3+0.3, which equals 0.6. See FIG. 11 for results. Accordingly, the combination of PHMB and EDTA(Na₄) unexpectedly has partial synergistic results for C. albicans. That is, embodiments of the combination of PHMB and EDTA(Na₄) provides results that are, unexpectedly, greater than the total effects of each agent operating by itself.

Rate Kill Assay—S. aureus

As discussed above, EDTA(Na₄) has a MIC of <0.03% (w/v) for S. aureus and PHMB has a MIC of <1.25 PPM for S. aureus. Accordingly, the following solutions were prepared:

| Composition | Concentration | | MIC |
|---|---|---|---|
| EDTA(Na₄) | 0.015 | wt % | 0.5 |
| PHMB | 0.625 | PPM | 0.5 |
| EDTA(Na₄) | 0.007 | wt % | 0.25 |
| PHMB | 0.31 | PPM | 0.25 |

| | | |
|---|---|---|
| EDTA(Na₄) + PHMB | 0.015 wt % + 0.625 PPM | 0.5 + 0.5 |
| EDTA(Na₄) + PHMB | 0.007 wt % + 0.31 PPM | 0.25 + 0.25 |

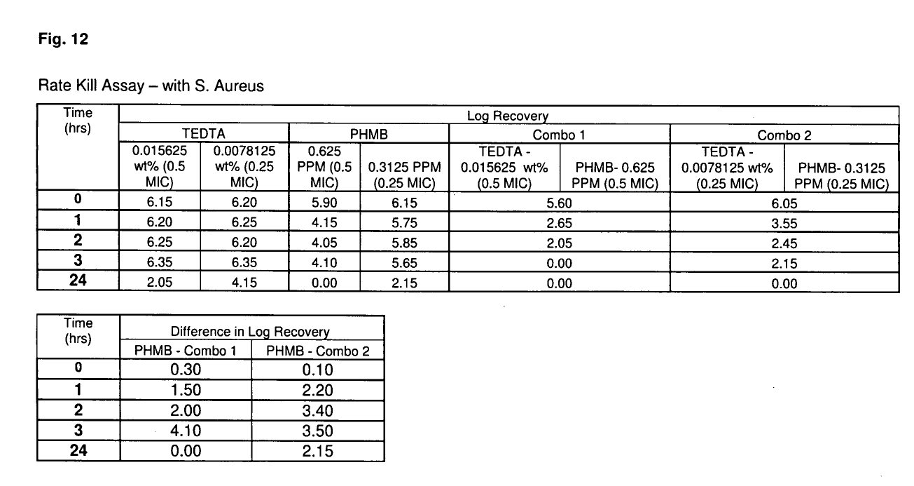

Each solution was then combined with S. aureus and the log recovery of the S. aureus was measured initially, after 0 hour, 1 hour, 2 hours, 3 hours and 24 hours. The difference in log recovery for the 0.5 MIC concentrations and for the 0.25 MIC concentrations is shown in FIG. 12. The data shows that EDTA(Na₄) and PHMB solutions are synergistic. That is, embodiments of the combination of EDTA(Na₄) and PHMB provides results that are, unexpectedly, greater than the total effects of each agent operating by itself.
Rate Kill Assay—P. aeruginosa

As discussed above, EDTA(Na₄) has a MIC of <0.25% (w/v) for P. aeruginosa, and PHMB has a MIC of <5 PPM for P. aeruginosa. Accordingly, the following solutions were prepared:

| Composition | Concentration | | MIC |
|---|---|---|---|
| EDTA(Na₄) | 0.125 | wt % | 0.5 |
| PHMB | 2.5 | PPM | 0.5 |
| EDTA(Na₄) | 0.0625 | wt % | 0.25 |
| PHMB | 1.25 | PPM | 0.25 |

| | | |
|---|---|---|
| EDTA(Na₄) + | 0.125 wt % + 2.5 PPM | 0.5 + 0.5 |
| PHMB | | |
| EDTA(Na₄) + | 0.0625 wt % + 1.25 PPM | 0.25 + 0.25 |
| PHMB | | |

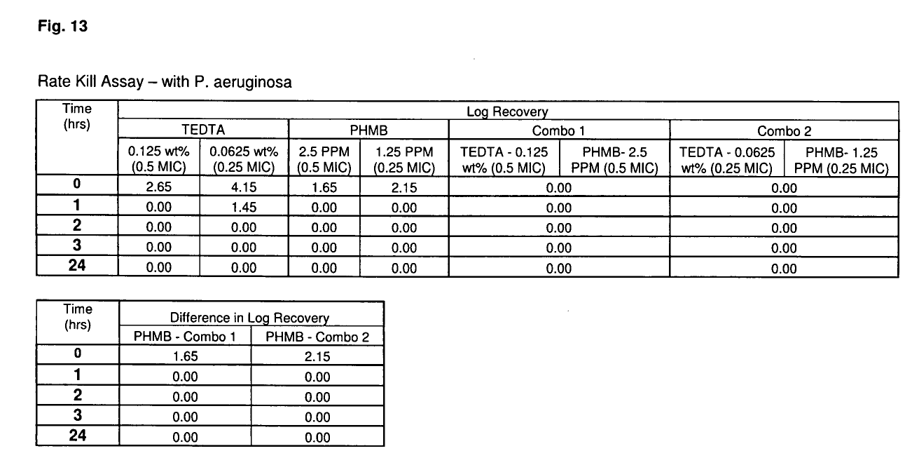

Each solution was then combined with P. aeruginosa and the log recovery of the P. aeruginosa was measured initially, after 0 hour, 1 hour, 2 hours, 3 hours and 24 hours. The difference in log recovery for the 0.5 MIC concentrations and for the 0.25 MIC concentrations is shown in FIG. 13. The data shows that EDTA(Na₄) and PHMB solutions are synergistic. That is, embodiments of the combination of EDTA(Na₄) and PHMB provides results that are, unexpectedly, greater than the total effects of each agent operating by itself.
Rate Kill Assay—C. albicans

As discussed above, EDTA(Na₄) has a MIC of <0.3125% (w/v) for C. albicans, and PHMB has a MIC of <1.25 PPM for C. albicans. Accordingly, the following solutions were prepared:

| Composition | Concentration | | MIC |
|---|---|---|---|
| EDTA(Na₄) | 0.007 | wt % | 0.25 |
| PHMB | 0.31 | PPM | 0.25 |
| EDTA(Na₄) | 0.0035 | wt % | 0.125 |
| PHMB | 0.15 | PPM | 0.125 |
| EDTA(Na₄) | 0.00525 | wt % | 0.1875 |
| PHMB | 0.2325 | PPM | 0.1875 |

| | | |
|---|---|---|
| EDTA(Na₄) + PHMB | 0.007 wt % + 0.31 PPM | 0.25 + 0.25 |
| EDTA(Na₄) + PHMB | 0.0035 wt % + 0.15 PPM | 0.125 + 0.125 |
| EDTA(Na₄) + PHMB | 0.00525 wt % + 0.2325 PPM | 0.1875 + 0.1875 |

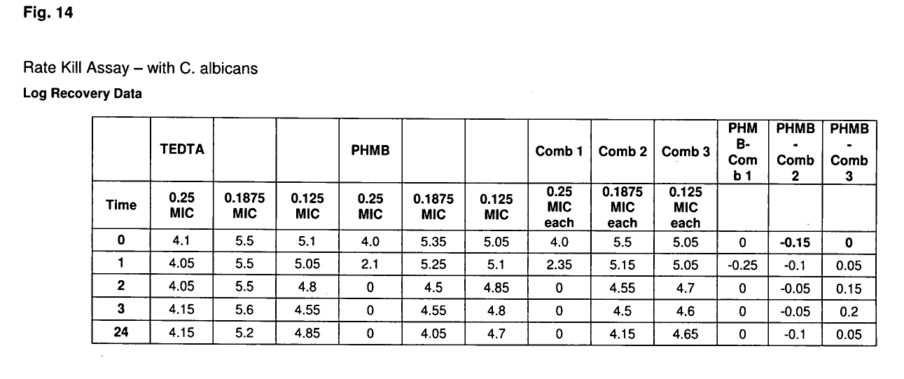

Each solution was then combined with C. albicans and the log recovery of the C. albicans was measured initially, after 0 hour, 1 hour, 2 hours, 3 hours and 24 hours. The difference in log recovery for the solutions is shown in FIG. 14. The data does not show that EDTA(Na₄) and PHMB solutions are synergistic. However, the data suggests the combination is very effective against C. ablicans with PHMB being the dominant component.

The synergistic effect (via rate kill assay and checkerboard titration for P. aeruginosa), partial synergistic effect (via checkerboard titration for S. Aureus and C. Albicans), and synergistic effect (via rate kill assay for S. Aureus) provides significant, practical advantages for uses of embodiments of the combination of PHMB and EDTA salt(s). Thus, embodiments of the present invention should prevent the overuse of broad-spectrum antibiotics and continued unnecessary catheter removal and replacement procedures.

### pH Experiments

Further experiments were conducted to measure the effects of pH on PHMB and EDTA formulations. In order to determine MIC (minimum inhibitory concentration) and MBC (minimum bactericidal concentration) a National Committee on Clinical Laboratory Standards (NCCLS) micro-dilution procedure was followed. According to the procedure each formulation must be exposed to 6 log concentration of organism or the highest achievable concentration. In the current protocol 100 μL of MHB was mixed with 90 μL of formulation and 10 μL of log 8 organism or the highest achievable concentration. The concentration of the formulation was adjusted to obtain the required concentration in the final solution. The mixture was incubated at 37 degree C. for 16-24 hrs. After 16-24 hours the absorbance value was read at 600 nm. The obtained data was corrected by subtracting the appropriate blanks. Finally, the wells having an absorbance >0.1 were marked + and <0.1 were marked −. The +symbol indicated growth while −symbol indicates no growth. The positive growth controls must have a corrective absorbance value of >0.5 and negative controls must have a corrected absorbance value of <0.1. In cases where the positive growth controls corrected absorbance is lower than 0.5, an alternate rule is utilized which is “absorbance<than 20% of positive growth control is marked as −growth, while absorbance≧than 20% of positive growth control is marked as +growth”. pH was adjusted to the stated value using NaOH or HCl.

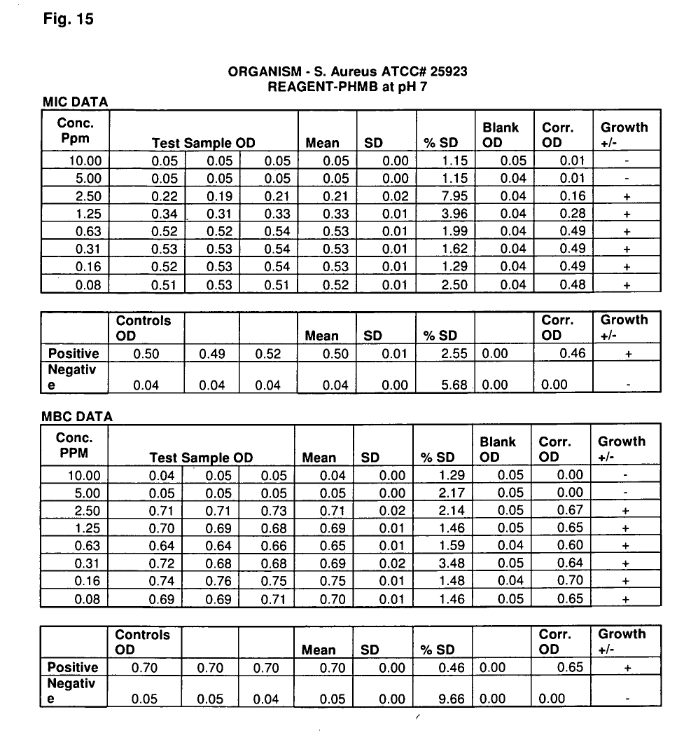

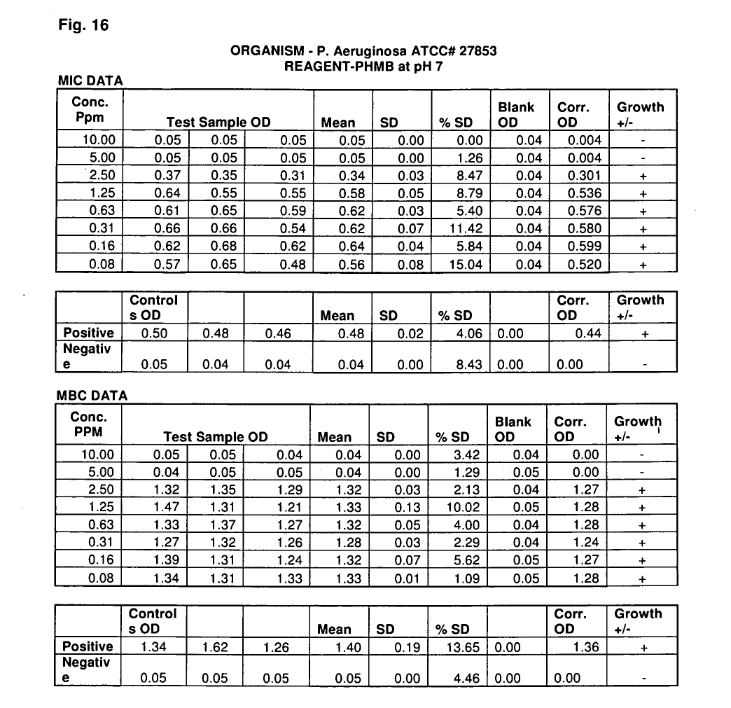

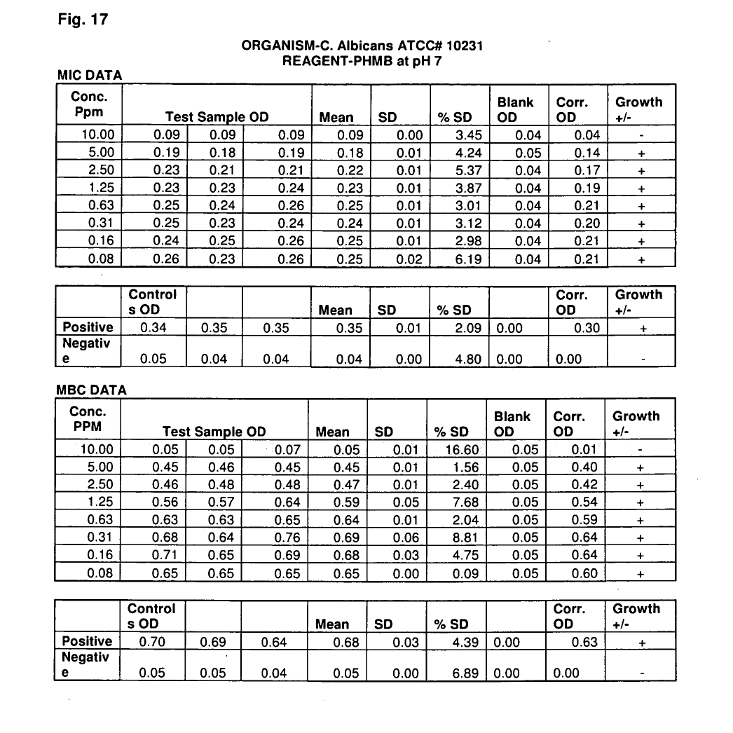

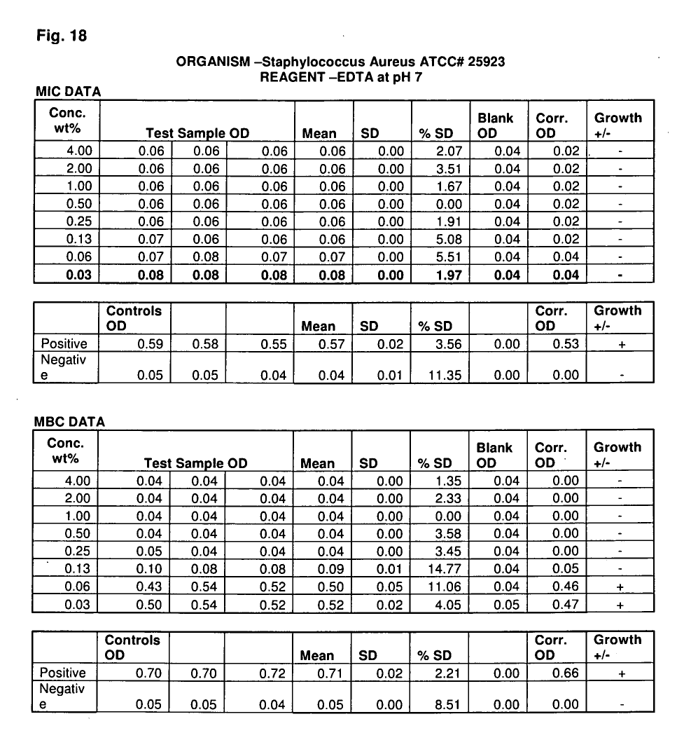

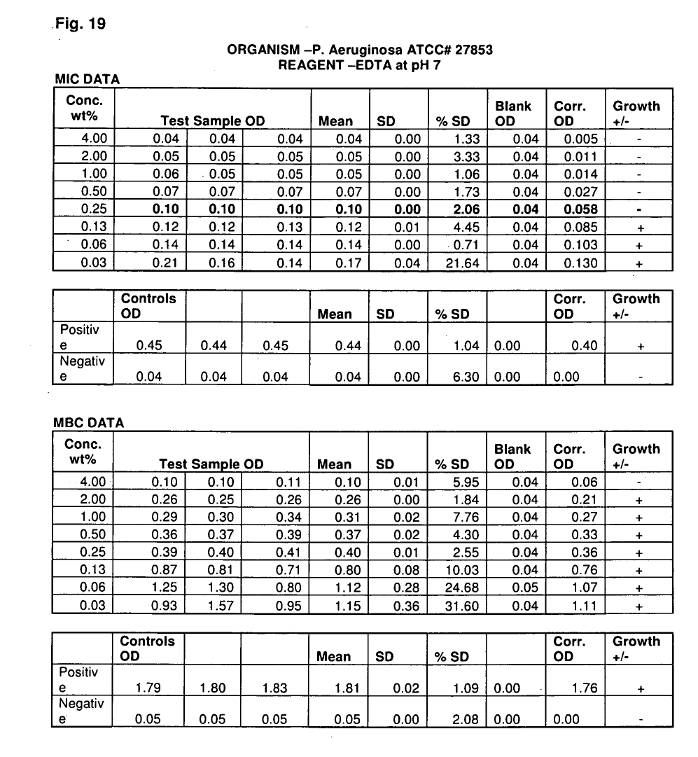

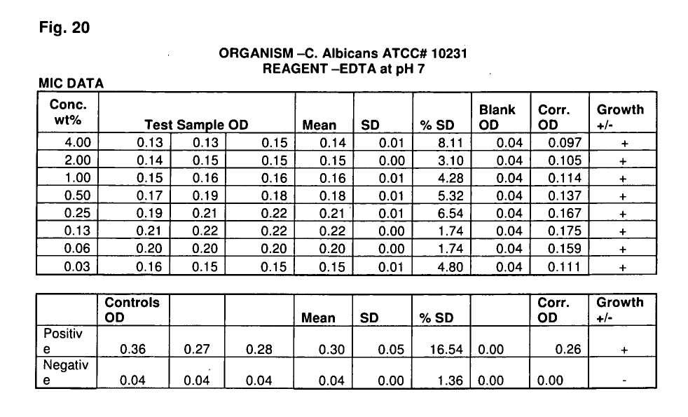

Staphylococcus aureus (Organism #25923), Pseudomonas aeruginosa (Organism #27853), and Candida Albicans (Organism #10231) was obtained from ATCC. PHMB was used (Avecia, Lot #1L15-038). EDTA, tetrasodium salt hydrate, was used (Alfa Aesar, Catalogue #A17385, Lot #J9570A). A 20 PPM PHMB solution in water was prepared at a pH of 7. A 8 wt % EDTA solution in water was prepared at a pH of 7. These solutions were then serially diluted as necessary to obtain the required concentrations. The MIC and MBC concentrations of PHMB and EDTA at a pH of 7 was found for each of S. aureus, P. aeruginosa, and C. albicans. See FIGS. 15-20 for results.

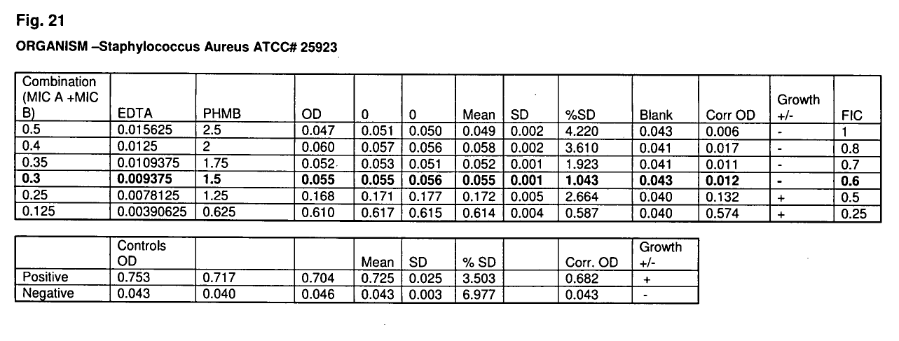

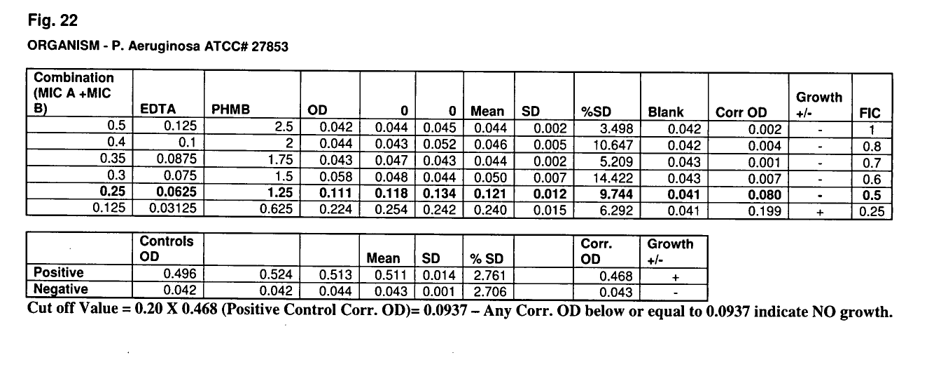

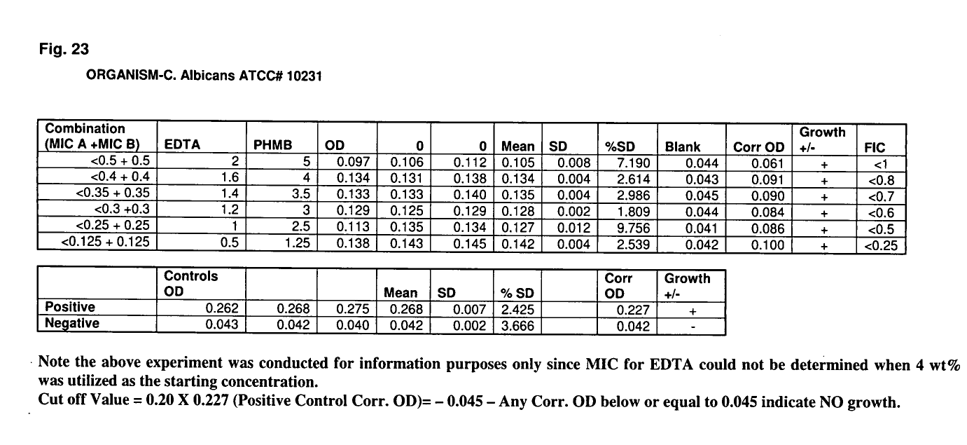

Based on the above, a further experiment conducted was a screening experiment using checkerboard titration to assess if the combinations at a pH of 7 fall within a range having an FIC index value of ≦1. The method used was a NCCLS micro-dilution procedure. The results of this experiment are shown in FIGS. 21-23. Based on the results the FIC index for PHMB and EDTA at a pH of 7 is 0.6 for S. aureus, 0.5 for P. aeruginosa and greater than 1 for C. albicans.

### Anticoagulant Experiments

Experiments were conducted to assess the anticoagulant capacities of PHMB, EDTA and combinations of PHMB and EDTA via a Prothrombin Time (PT) Assay. A PT assay (TM-4339-063) was conducted using a Coagulation Analyzer to obtain PT instead of manually recording the PT.

Tetrasodium EDTA (TEDTA) was used (Alfa Aesar, Catalog #A17385, Lot #J9570A). PHMB was used (Arch Biocides, Catalogue #84312, Lot #1L15-038). TriniCHECK 1 (Normal Control) was used (Trinity Biotech). TriniCHECK 2 (Abnormal Control) was used (Trinity Biotech). A KC4 Amelung Coagulizer was used (Trinity Biotech).

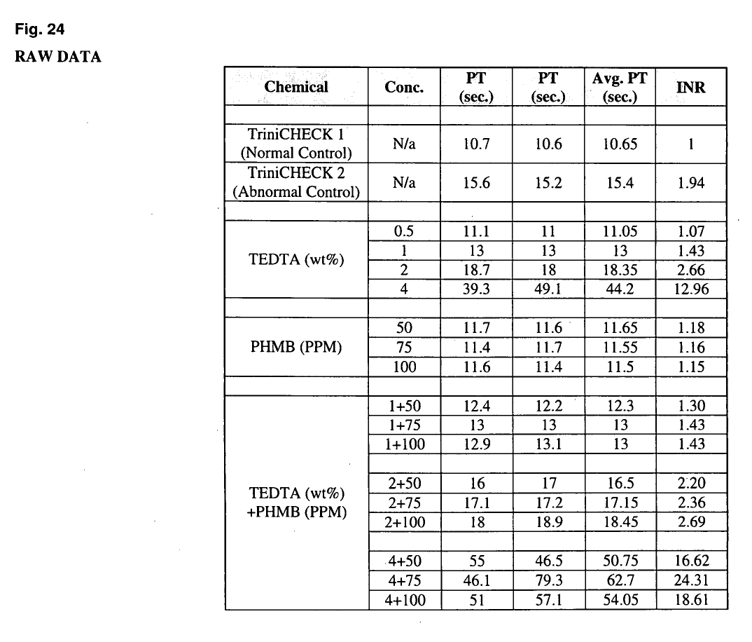

FIG. 24 shows the results (raw data) of the PT assay. The concentrations stated in the concentration column are the final concentrations of the reagents. TriniCHECK 1 is a normal control that provides the PT time in the range of what a normal blood sample would take to coagulate. TriniCHECK 2 is an abnormal control that provides the PT time above the range of what a normal blood sample would take to coagulate. INR (International Normalized Ratio) is a system established by the World Health Organization (WHO) and the International Committee on Thrombosis and Hemostasis for reporting the results of blood coagulation (clotting) tests. INR is calculated as:

INR=(PT_{test sample}/PT_{normal control})^{ISI}

ISI (International Sensitivity Index) indicates the sensitivity of individual thromboplastin. The value of ISI utilized herein was 1.89.

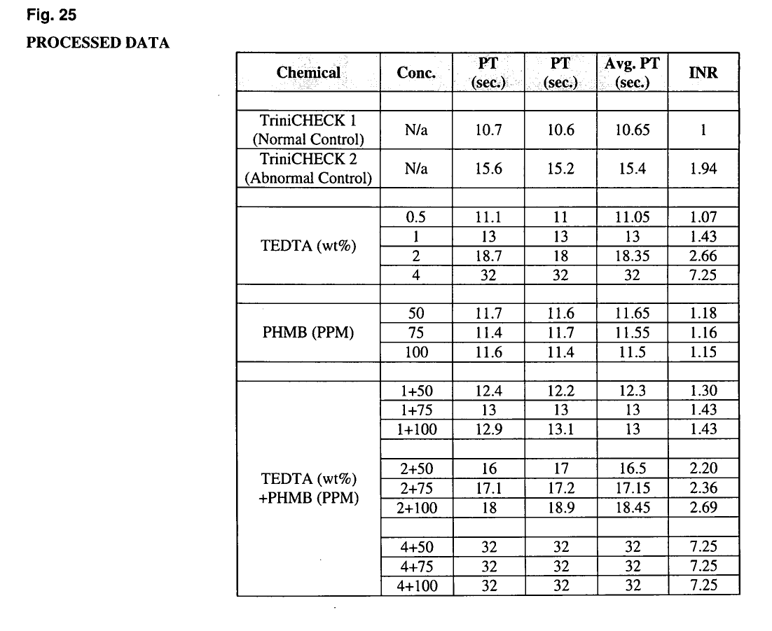

FIG. 25 shows the results (processed data) of the PT assay. All the PTs greater than 3×the TriniCHECK 1 (normal control) were replaced with 32 seconds. This was done for the following reasons: Instrument used does not provide reproducible readings at PTs greater than 45 seconds; PTs greater than 3×the normal control results in INR greater than 6 if the ISI is 1.89. Any INR value higher than 5.5 indicates very high anticoagulant capacity and any higher value is of very little or no clinical significance; and for better assessment of data.

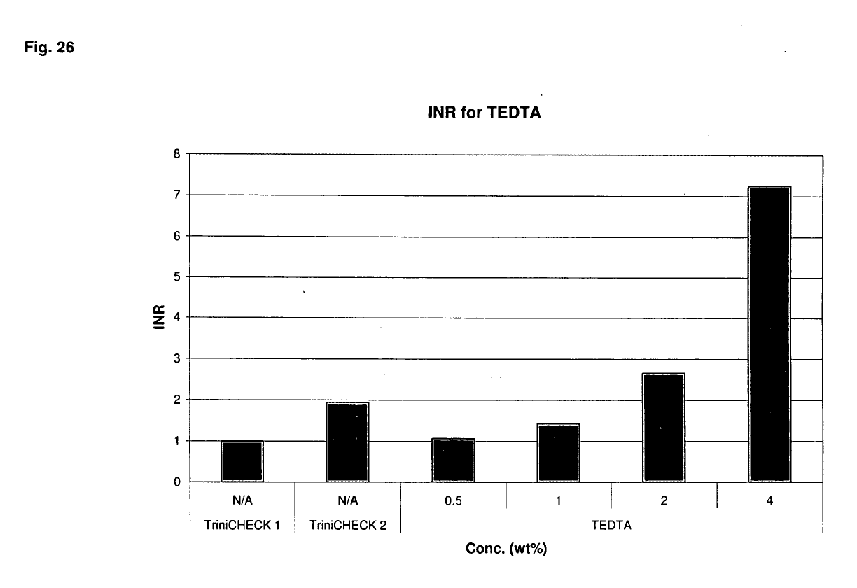

FIG. 26 shows the graph of the International Normalized Ratio (INR) for TEDTA from a Prothrombin Time (PT) Assay. From FIG. 26 it is evident that (within the tested range) that at a concentration of TEDTA of 4 wt %, the INR is greater than 7.25.

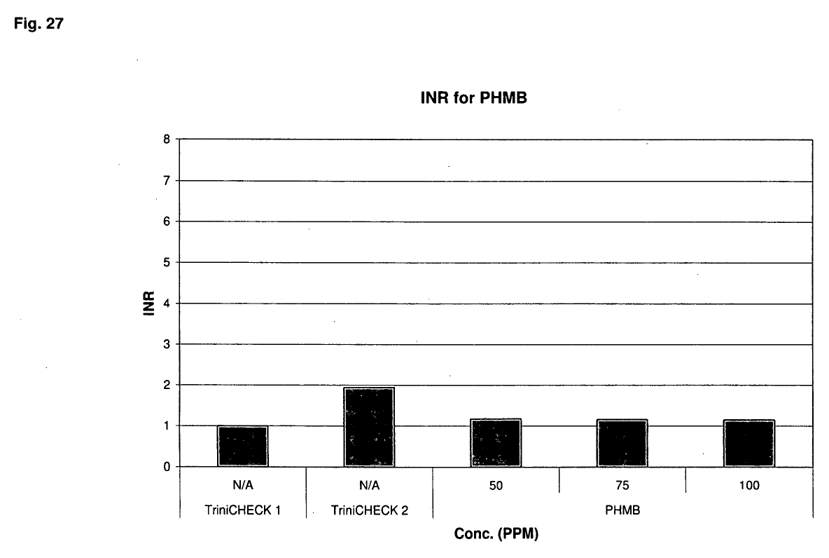

FIG. 27 shows the graph of the International Normalized Ratio (INR) for PHMB from a Prothrombin Time (PT) Assay. From FIGS. 24,25 & 27 is it evident that (within the tested range) than an increase in concentration of PHMB results in no significant increase in INR.

FIG. 28 shows the graph of the International Normalized Ratio (INR) for combined TEDTA and PHMB formulations from a Prothrombin Time (PT) Assay. From FIG. 28, and comparing results from FIGS. 26 and 27, it is evident that (within the tested range) that the addition of PHMB does not significantly promote or enhance the anticoagulant activity of TEDTA, but also does not negatively affect the anticoagulant activity of TEDTA. Accordingly, TEDTA (4 wt %) mixed with PHMB at 50, 75 or 100 ppm provides very good anticoagulant activity.

From the foregoing, it should be clear that the present disclosure may be embodied in forms other than those discussed above; the scope of the present disclosure should be determined by the following claims and not the detailed discussion presented above.

1. A disinfectant composition comprising PHMB and an EDTA salt in solution, wherein the PHMB is at a concentration of at least 0.1 PPM and less than 400 PPM, the EDTA salt is at a concentration of at least 0.0125% (w/v) and less than 12.0% (w/v), and the disinfectant composition has pH a greater than 7.

2. The composition of claim 1, wherein the solution comprises between 0 and 10% (v/v) ethanol and water.

3. The composition of claim 1, wherein the solution comprises saline.

4. The composition of claim 1, wherein the PHMB is at a concentration of at least 5 PPM and less than 200 PPM, wherein the EDTA salt is a tetrasodium salt at a concentration of at least 0.25% (w/v) and less than 8% (w/v), and wherein the disinfectant composition has a pH of greater than 9.0.

5. A disinfectant composition comprising PHMB and an EDTA salt in a dry or partially hydrated formulation that, upon reconstitution with a solution, forms a disinfectant composition wherein the PHMB is at a concentration of at least 0.1 PPM and less than 400 PPM, the EDTA salt is at a concentration of at least 0.0125% (w/v) and less than 12.0% (w/v), and the disinfectant composition has a pH greater than 7.

6. A disinfectant composition comprising PHMB and an EDTA salt in solution, wherein the PHMB is at a concentration of at least 0.1 PPM and less than 400 PPM, the EDTA salt is at a concentration of at least 0.0125% (w/v) and less than 12.0% (w/v), the disinfectant composition has a pH greater than 7, the disinfectant composition is packaged in a sterile, non-pyrogenic form, the solution is water, and the disinfectant composition has an osmolarity of from 240-500 mOsM/Kg.

7. A lock flush composition comprising PHMB and an EDTA salt in solution, wherein the PHMB is at a concentration of at least 0.1 PPM and less than 400 PPM, the EDTA salt is at a concentration of at least 0.0125% (w/v) and less than 12.0% (w/v), the disinfectant composition has a pH greater than 7, the lock flush composition is packaged in a sterile, non-pyrogenic form, and the lock flush composition is biocompatible for use in in-dwelling access catheters, urinary catheters, nasal tubes and throat tubes.

8. The composition of claim 7, wherein the PHMB is at a concentration of at least 5 PPM and less than 200 PPM, wherein the EDTA salt is a tetrasodium salt at a concentration of at least 0.25% (w/v) and less than 8% (w/v), and wherein the disinfectant composition has a pH of greater than 9.0.

9. A method for disinfecting a catheter by contacting the catheter with a disinfectant solution comprising PHMB and an EDTA salt in solution, PHMB is at a concentration of at least 0.1 PPM and less than 400 PPM, the EDTA salt is at a concentration of at least 0.0125% (w/v) and less than 12.0% (w/v), the disinfectant composition has a pH greater than 7.

10. The method of claim 9, wherein the solvent is water.

11. The method of claim 9, wherein the PHMB is at a concentration of at least 5 PPM and less than 200 PPM, wherein the EDTA salt is a tetrasodium salt at a concentration of at least 0.25% (w/v) and less than 8% (w/v), and wherein the disinfectant composition has a pH of >9.0.

12. The method of claim 9, wherein contacting the catheter with the disinfectant solution is accomplished by locking, flushing or coating the catheter with the disinfectant solution.

13. The method of claim 9, wherein the catheter is selected from the group consisting of peripherally inserted catheters, central venous catheters, peritoneal catheters, hemodialysis catheters and urological catheters.

14. A method for disinfecting a catheter comprising:
introducing a disinfectant solution into an interior lumen of the catheter, wherein the disinfectant solution comprises PHMB and an EDTA salt in solution, wherein the PHMB is at a concentration of at least 0.1 PPM and less than 400 PPM, the EDTA salt is at a concentration of at least 0.0125% (w/v) and less than 12.0% (w/v), the disinfectant composition has a pH greater than 7;
holding the disinfectant solution within the lumen for a selected period of time; and
removing the disinfectant solution from the interior lumen.

15. An anticoagulant composition comprising PHMB and an EDTA salt in solution, wherein the PHMB is at a concentration of at least 0.1 PPM and less than 400 PPM, the EDTA salt is at a concentration of at least 0.0125% (w/v) and less than 12.0% (w/v), and the disinfectant composition has pH a greater than 7.

16. The composition of claim 15, wherein the PHMB is at a concentration of at least 5 PPM and less than 200 PPM, wherein the EDTA salt is a tetrasodium salt at a concentration of at least 0.25% (w/v) and less than 8% (w/v).
